SEQUENCE LISTING (21 sequences)

SEQ ID NO: 1 - DNA, 348 bp, Mus musculus
gcggtccagctgcagcagtctggacctgagctggaaaagcctggcgcttcagtgaagatttcctgcaaggcttctggttactcattcactggctacaatatgaactgggtgaagcagaataatggaaagagccttgagtggattggaaatattgatccttattatggtggtactacctacaaccggaagttcaagggcaaggccacattgactgtagacaaatcctccagcacagcctacatgcagctcaagagtctgacatctgaggactctgcagtctattactgtgcaagatcggtcggccctatggactactggggtcaaggaacctcagtcaccgtctcttct

SEQ ID NO: 2 - protein, 116 aa, Mus musculus
AVQLQQSGPELEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSS

SEQ ID NO: 3 - DNA, 324 bp, Mus musculus
gacatccagatgactcagtctccagcctccctatctgcatctgtgggagagactgtcaccatcacatgtcgaacaagtgaaaatgtttacagttatttggcttggtatcagcagaaacagggaaaatctcctcagctcctggtctcttttgcaaaaaccttagcagaaggtgtgccatcaaggttcagtggcagtggatcaggcacacagttttctctgaagatcagcagcctgcagcctgaagattctggaagttatttctgtcaacatcattccgataatccgtggacgttcggtggaggcaccgaactggagatcaaacga

SEQ ID NO: 4 - protein, 108 aa, Mus musculus
DIQMTQSPASLSASVGETVTITCRTSENVYSYLAWYQQKQGKSPQLLVSFAKTLAEGVPSRFSGSGSGTQFSLKISSLQPEDSGSYFCQHHSDNPWTFGGGTELEIKR

SEQ ID NO: 5 - protein, 446 aa, Artificial Sequence
  Description of Artificial Sequence Synthetic construct heavy chain A2 polypeptide
AVQLQQSGPELEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  Description of Artificial Sequence Synthetic construct light chain A2 polypeptide
DIQMTQSPASLSASVGETVTITCRTSENVYSYLAWYQQKQGKSPQLLVSFAKTLAEGVPSRFSGSGSGTQFSLKISSLQPEDSGSYFCQHHSDNPWTFGGGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 330 aa, Artificial Sequence
  Description of Artificial Sequence Synthetic construct constant heavy chain polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 106 aa, Artificial Sequence
  Description of Artificial Sequence Synthetic construct constant light chain polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 446 aa, Artificial Sequence
  Description of Artificial Sequence Synthetic construct heavy chain A4 polypeptide
AVQLQQSGPELEKPGASVKISCKASGYSFTGYNMNWVKQNNGKSLEWIGNIDPYYGGTTYNRKFKGKATLTVDKSSSTAYMQLKSLTSEDSAVYYCARSVGPMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence; identical to SEQ ID NO: 6
  Description of Artificial Sequence Synthetic construct light chain A4 polypeptide

SEQ ID NO: 11 - protein, 446 aa, Artificial Sequence
  Description of Artificial Sequence Synthetic construct heavy chain B2 polypeptide
AVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, Artificial Sequence
  Description of Artificial Sequence Synthetic construct light chain B2 polypeptide
DIQMTQSPSSLSASVGDRVTITCRTSENVYSYLAWYQQKPGKAPKLLVSFAKTLAEGVPSRFSGSGSGTDFTLTISSLQPEDFATYFCQHHSDNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, Artificial Sequence
  Description of Artificial Sequence Synthetic construct heavy chain B4 polypeptide
AVQLVQSGAEVKKPGSSVKVSCKASGYSFTGYNMNWVRQAPGQGLEWMGNIDPYYGGTTYNRKFKGRVTLTVDKSSSTAYMELSSLRSEDTAVYYCARSVGPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 214 aa, Artificial Sequence; identical to SEQ ID NO: 12
  Description of Artificial Sequence Synthetic construct light chain B4 polypeptide

SEQ ID NO: 15 - protein, 5 aa, Artificial Sequence
  Description of Artificial Sequence Synthetic CDR1 heavy chain (H1) peptide
GYNMN

SEQ ID NO: 16 - protein, 17 aa, Artificial Sequence
  Description of Artificial Sequence Synthetic CDR2 heavy chain (H2) peptide
NIDPYYGGTTYNRKFKG

SEQ ID NO: 17 - protein, 7 aa, Artificial Sequence
  Description of Artificial Sequence Synthetic CDR3 heavy chain (H3) peptide
SVGPMDY

SEQ ID NO: 18 - protein, 11 aa, Artificial Sequence
  Description of Artificial Sequence Synthetic CDR1 light chain (L1) peptide
RTSENVYSYLA

SEQ ID NO: 19 - protein, 7 aa, Artificial Sequence
  Description of Artificial Sequence Synthetic CDR2 light chain (L2) peptide
FAKTLAE

SEQ ID NO: 20 - protein, 9 aa, Artificial Sequence
  Description of Artificial Sequence Synthetic CDR3 light chain (L3) peptide
QHHSDNPWT

SEQ ID NO: 21 - protein, 13 aa, Artificial Sequence
  Description of Artificial Sequence Synthetic alternative CDR2 heavy chain (H2b) peptide
NIDPYYGGTTYNR

(19) United States
(12) Patent Application Publication
(10) Pub. No.: US 2015/0231242 A1
(43) Pub. Date: Aug. 20, 2015

(54) COMBINATION OF CD37 ANTIBODIES WITH BENDAMUSTINE

(71) Applicant: Boehringer Ingelheim International GmbH, Ingelheim am Rhein (DE)

(72) Inventors: Karl-Heinz HEIDER, Stockerau (AT); Petra BLUM, Biberach an der Riss (DE)

(21) Appl. No.: 14/703,287

(22) Filed: May 4, 2015

Related U.S. Application Data
(63) Continuation of application No. 13/870,487, filed on Apr. 25, 2013.

(30) Foreign Application Priority Data
Apr. 26, 2012 (EP) 12165660.7
May 16, 2012 (EP) 12168397.3

(51) Int. Cl. A61K 39/395; A61K 31/4184

(52) U.S. Cl. CPC A61K 39/39558; A61K 31/4184; A61K 2039/545

(57) ABSTRACT

The present invention relates to immunotherapies that are based on depletion of CD37-positive cells such as B-cells. The present invention provides methods for reduction of CD37-positive cells such as B-cells in an individual/patient using a combination of CD37 antibody/antibodies and bendamustine. The combination of CD37 antibodies and bendamustine is shown to have a synergistic effect. The application further provides materials and methods for treatment of diseases involving aberrant B-cell activity.

### TECHNICAL FIELD

The present invention relates to immunotherapies that are based on depletion of CD37-positive cells such as B-cell cells. In particular, the present invention relates to a combination of CD37 antibodies, especially A2 and B2, with chemotherapy, especially bendamustine for use in such therapies, e.g. in the treatment of B-cell malignancies, other CD37-positive malignancies, and autoimmune conditions.

### BACKGROUND OF THE INVENTION

Immunotherapy using monoclonal antibodies (mAbs) has emerged as a safe and selective method for the treatment of cancer and other diseases. In particular, the role of monoclonal antibodies in therapies that are based on B-cell depletion, e.g. in the treatment of B-cell malignancies, has expanded since the introduction of rituximab (Rituxan®), an antibody that is directed against the CD20 antigen on the B-cell surface. Numerous studies have confirmed the efficacy of rituximab as a single agent and in combination therapy in low-grade NHL. Frontline therapy with rituximab added to the combination of cyclophosphamide, doxorubicin, vincristine, and prednisolone (CHOP) significantly improves the outcome for patients with advanced-stage follicular lymphoma compared with therapy with CHOP alone (Hiddemann W, et al. Blood 2005; 106: 3725-3732 (2005)) The addition of rituximab to a combination of fludarabine, cyclophosphamide, mitoxantrone (FCM) significantly increases the response rate and prolongs survival as compared with FCM alone in patients with relapsed and refractory follicular and mantle cell lymphomas (Forstpointner R, et al., Blood, 2004; 104: 3064-3071).

However, only a subset of patients responds to therapy and the majority of those eventually relapse following rituximab treatment. Therefore, there is a need to find immunotherapies with higher efficacy than antibodies that are directed against the CD20 antigen (rituximab).

### SUMMARY OF THE INVENTION

The invention describes CD37 antibodies, preferably A2 and B2, used in combination with bendamustine. This combination surprisingly results in a synergistic anti-tumor effect. The two therapeutic agents, CD37 antibody and bendamustine, may be administered simultaneously, optionally as a component of the same pharmaceutical preparation, or bendamustine may be administered before or after administration of the CD37 antibody.

In accordance with the invention, there are provided novel combinations of anti-CD37 antibodies as described in the present invention with bendamustine. Accordingly, the combination of anti-CD37 antibodies of the present invention and bendamustine are used to treat patients suffering from B-cell malignancies.

A high degree of tumor cell killing in patients with B-cell malignancies, e.g. CLL and B-NHL, is considered advantageous for the treatment of those patients and is considered to translate into increased clinical benefit for patients treated with such an agent. CD37 antibodies such as A2 in combination with bendamustine display a high degree of tumor cell apoptosis in in vitro assays with Ramos and Raji lymphoma cells. The pro-apoptotic effect of the combination of CD37 mAb and bendamustine is superior to the effect of the individual agents alone (see data disclosed in this application). Apoptosis induction is considered a surrogate parameter for cell death and thus ultimately will lead to tumor cell kill and depletion. This superior efficacy of A2 in combination with bendamustine is especially evident in FIGS. 1 and 2 and is clearly superior to that of the individual agents alone.

The benefit of a combination treatment with CD37 antibodies, especially mAbs A2 or B2, and a chemotherapeutic agent such as bendamustine is further demonstrated in clinical trials, which compare the efficacy of bendamustine monotherapy against the efficacy of a combination of bendamustine and CD37 antibodies, especially mAb A2 or B2. The trial is performed in a randomized fashion, e.g. the patients are equally assigned to the two different treatment arms of the study in a blinded and randomized fashion. The response to treatment is defined by standardized response criteria for the respective indication. The efficacy of the treatment is assessed by surrogate parameters like progression free survival (PFS). A clinically relevant therapeutic effect is the prolongation of PFS by 50% with bendamustine and A2 or B2 compared to bendamustine alone (e.g. 27 months PFS compared to 18 months) for patients with relapsed chronic lymphocytic leukemia.

To be used in therapy, the CD37 antibody is included into pharmaceutical compositions appropriate to facilitate administration to animals or humans. Typical formulations of the CD37 antibody molecule can be prepared by mixing the CD37 antibody molecule with physiologically acceptable carriers, excipients or stabilizers, in the form of lyophilized or otherwise dried formulations or aqueous solutions or aqueous or non-aqueous suspensions.

Pharmaceutically acceptable carriers and adjuvants for use with CD37 antibodies according to the present invention include, for example, ion exchangers, alumina, aluminum stearate, lecithin, serum proteins, buffer substances, water, salts or electrolytes and cellulose-based substances.

Carriers, excipients, modifiers or stabilizers are nontoxic at the dosages and concentrations employed. They include buffer systems such as phosphate, citrate, acetate and other inorganic or organic acids and their salts; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, oligosaccharides or polysaccharides and other carbohydrates including glucose, mannose, sucrose, trehalose, dextrins or dextrans; chelating agents such as EDTA; sugar alcohols such as, mannitol or sorbitol; salt-forming counter-ions such as sodium; metal complexes (e.g. Zn-protein complexes); and/or ionic or non-ionic surfactants such as TWEEN™ (polysorbates), PLURONICS™ or fatty acid esters, fatty acid ethers or sugar esters. Also organic solvents can be contained in the antibody formulation such as ethanol or isopropanol. The excipients may also have a release-modifying or absorption-modifying function. This is not a complete list of possible pharmaceutically acceptable carriers and adjuvants, and one of ordinary skilled in the art would know other possibilities, which are replete in the art.

As further explained in Example 3 below, in one embodiment the CD37 antibody A2 is formulated in a vehicle containing 25 mM Na-citrate, 115 mM NaCl and 0.04% Tween 80, pH 6.0 and diluted with PBS.

The CD37 antibody molecules may also be dried (freeze-dried, spray-dried, spray-freeze dried, dried by near or supercritical gases, vacuum dried, air-dried), precipitated or crystallized or entrapped in microcapsules that are prepared, for example, by coacervation techniques or by interfacial polymerization using, for example, hydroxymethylcellulose or gelatin and poly-(methylmethacylate), respectively, in colloidal drug delivery systems (for example, liposomes, albumin microspheres, microemulsions, nano-particles and nanocapsules), in macroemulsions or precipitated or immobilized onto carriers or surfaces, for example by pcmc technology (protein coated microcrystals). Such techniques are known in the art.

Naturally, the pharmaceutical compositions/formulations to be used for in vivo administration must be sterile; sterilization may be accomplished be conventional techniques, e.g. by filtration through sterile filtration membranes.

It may be useful to increase the concentration of the CD37 antibody to come to a so-called high concentration liquid formulation (HCLF); various ways to generate such HCLFs have been described.

The CD37 antibody molecule may also be contained in a sustained-release preparation. Such preparations include solid, semi-solid or liquid matrices of hydrophobic or hydrophilic polymers, and may be in the form of shaped articles, e.g. films, sticks or microcapsules and may be applied via an application device. Examples of sustained-release matrices include polyesters, hydrogels (for example, poly(2-hydroxyethyl-methacrylate) or sucrose acetate butyrate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilization from acidic solutions, controlling moisture content, using appropriate additives, and developing specific polymer matrix compositions.

The CD37 antibody molecule, especially A2 and B2, can be incorporated also in other application forms, such as dispersions, suspensions or liposomes, tablets, capsules, powders, sprays, transdermal or intradermal patches or creams with or without permeation enhancing devices, wafers, nasal, buccal or pulmonary formulations, or may be produced by implanted cells or—after gene therapy—by the individual's own cells.

A CD37 antibody molecule, especially A2 and B2, may also be derivatized with a chemical group such as polyethylene glycol (PEG), a methyl or ethyl group, or a carbohydrate group. These groups may be useful to improve the biological characteristics of the antibody, e.g. to increase serum half-life or to increase tissue binding.

The preferred mode of application is parenteral, by infusion or injection (intravenous, intramuscular, subcutaneous, intraperitoneal, intradermal), but other modes of application such as by inhalation, transdermal, intranasal, buccal, oral, may also be applicable.

For therapeutic use, the compounds may be administered in a therapeutically effective amount in any conventional dosage form in any conventional manner. Routes of administration include, but are not limited to, intravenously, intramuscularly, subcutaneously, intrasynovially, intrathecally by infusion, sublingually, transdermally, orally, topically or by inhalation, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). A therapeutically effective amount can be determined by a skilled artisan based upon such factors as weight, metabolism, and severity of the affliction etc.

Preferably the active compound is dosed at about 0.01 μg to about 500 mg per kilogram of body weight at least once per treatment cycle, e.g. on a weekly basis (0.01 μg to 500 mg per kilogram of body weight). More preferably the active compound is dosed at about 0.01 mg to 40 mg per kilogram of body weight at least once per treatment cycle.

For the prevention or treatment of disease, the appropriate dosage of antibody will depend on the type of disease to be treated, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 0.01 μg/kg to 40 mg/kg of CD37 antibody, especially of A2 and B2, is an initial candidate dosage for administration to the patient, whether, for example, by one or more separate administrations, or by infusion such as continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays, e.g. by determining the extent of B-cell depletion (e.g. using flow cytometry).

For A2, the estimated weekly dose for a 70 kg human is in the range of 1 mg to 2800 mg, preferably 1 mg to 400 mg weekly or 2 mg to 800 mg every 2 weeks. The estimated human weekly dose for B2 for a 70 kg human is in the range of 1 mg to 2800 mg, preferably 1 mg to 1000 mg, e.g. 100 mg to 385 mg weekly or 200 mg to 770 mg every two weeks for a 70 kg person.

Treatment cycle: The treatment cycle is a time period of between 1 to 6 weeks, preferably 3 to 4 weeks, most preferably 4 weeks, wherein the patient receives at least one dose of the CD37 antibody and at least one dose of bendamustine.

For CLL a preferred treatment cycle scheme lasts for a time period of 4 weeks, whereby bendamustine is preferably administered at a dose of 100 mg/m² body surface preferably on day 1 and 2 and whereby at least one CD37 antibody, preferably A2 or B2, is administered at a dose as described above either before, after or simultaneously with the bendamustine administration. Simultaneously hereby means on the same day. Simultaneously furthermore may mean within six hours of each other or within one hour of each other or with the same injection. Furthermore, another preferred treatment cycle scheme for CLL comprises additional administration(s) of CD37 antibody in between, for example in the middle of the treatment cycle at about 2 weeks.

For NHL a preferred treatment cycle scheme lasts for a time period of 3 weeks, whereby bendamustine is preferably administered at a dose of 120 mg/m² body surface preferably on day 1 and 2 and whereby at least one CD37 antibody, preferably A2 or B2, is administered at a dose as described above either before, after or simultaneously with the bendamustine administration. Simultaneously hereby means on the same day. Simultaneously furthermore may mean within six hours of each other or within one hour of each other or with the same injection. Furthermore, another preferred treatment cycle scheme for NHL comprises additional administration(s) of CD37 antibody in between, for example once a week, thus resulting in several, preferably 3 to 4 administrations of CD37 antibody per treatment cycle.

Bendamustine is preferably dosed on two consecutive days (e.g. d1+d2) of a treatment cycle, which is preferably 3-4 weeks (=21-28 days) long. The dose for bendamustine ranges between 50-150 mg/m2 body surface on 2 treatment days of a 3 to 4 week long treatment cycle. Preferably the dose ranges between 70-120 mg/m2 body surface or between 100-150 mg/m2 body surface on d1+2 of a treatment cycle. For the treatment of a CLL patient bendamustine is preferably administered at a dosage of 100 mg/m2 body surface on days 1 and 2 of the treatment cycle (e.g. 3-4 weeks, preferably 4 weeks). For the treatment of a NHL patient bendamustine is preferably administered at a dosage of 120 mg/m2 body surface on days 1 and 2 of the treatment cycle (e.g. 3-4 weeks, preferably 3 weeks). Furthermore preferred is a dose in the range of 60-70 mg/m2 body surface on d1+2 of a treatment cycle. But also a one-time administration of bendamustine may be administered per treatment cycle with a somewhat higher dose (e.g. 140-400 mg/m2).

The bendamustine dose is administered preferably on day 1 and on day 2 of a 3-4 week treatment cycle. Furthermore, preferred is the administration of bendamustine on the 2 days following a CD37 antibody administration (e.g. day 1=CD37 administration in any of the dosages as described above, days 2+3=bendamustine administration in any of the dosages as described above) of a 3-4 week treatment cycle.

The bendamustine dose may be administered by any way, e.g. infusion, parenteral or oral administration.

The “therapeutically effective amount” of the antibody to be administered is the minimum amount necessary to prevent, ameliorate, or treat a disease or disorder.

CD37-positive malignancies include, without limitation, all malignancies that express CD37. B-cell malignancies belong to the group of CD37-positive malignancies. B-cell malignancies include, without limitation, B-cell lymphomas (e.g. various forms of Hodgkin's disease, B-cell non-Hodgkin's lymphoma (NHL) and related lymphomas (e.g. Waldenström's macroglobulinaemia (also called lymphoplasmacytic lymphoma or immunocytoma) or central nervous system lymphomas), leukemias (e.g. acute lymphoblastic leukemia (ALL), chronic lymphocytic leukemia (CLL; also termed B-cell chronic lymphocytic leukemia BCLL), hairy cell leukemia and chronic myelogenous leukemia). Additional B-cell malignancies include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt's lymphoma/leukemia, grey zone lymphoma, B-cell proliferations of uncertain malignant potential, lymphomatoid granulomatosis, and post-transplant lymphoproliferative disorder. In addition, CD37-positive malignancies include, without limitation, T-cell lymphomas, multiple myelomas, and acute lymphocytic leukemias.

The CD37 antibody may be administered alone or in combination with adjuvants that enhance the stability, facilitate administration of pharmaceutical compositions containing them in certain embodiments, provide increased dissolution or dispersion, increase activity, provide adjunct therapy, and the like. Advantageously, such combinations may utilize lower dosages of the active ingredient, thus reducing possible toxicity and adverse side effects.

### BRIEF DESCRIPTION OF THE FIGURES

FIG. 1 shows the apoptosis induction on Raji cells. The cells were treated with bendamustine (200 μM or 400 μM), A2 (10 μg/ml) and a combination of A2 and bendamustine, for 48 hours. The percentage of apoptotic cells is displayed as AnnexinV positive cells corrected for background apoptosis in the presence of medium control and DMSO. For the combination treatment, the additive effect of the two individual treatments was calculated (A2+bendamustine calculated) and compared to the value measured by FACS analysis (A2+bendamustine measured). Bars represent mean values of three independent experiments, standard deviation is indicated.

FIG. 2 shows the apoptosis induction on Ramos cells. The cells were treated with bendamustine (100 μM or 400 μM), A2 (10 μg/ml) and a combination of A2 and bendamustine for 48 hours. The percentage of apoptotic cells is displayed as AnnexinV positive cells corrected for background apoptosis in the presence of medium control and DMSO. For the combination treatment, the additive effect of the two individual treatments was calculated (A2+bendamustine calculated) and compared to the value measured by FACS analysis (A2+bendamustine measured). Bars represent mean values of three independent experiments, standard deviation is indicated.

FIG. 3 shows the statistical analysis of interaction contrasts. Apoptotic effects were quantified by mean differences and their two-sided 95% confidence intervals. Interaction contrasts are considered synergistically for p-values<0.05.

FIG. 4 shows the DOHH2 tumor growth kinetics. DOHH2 tumor-bearing mice were treated with Antibody A2, bendamustine or with the combination of Antibody A2 and bendamustine. Median tumor volumes are plotted over time. Day 1 was the first day, day 16 the last day of the experiment. The symbols on the top denote the days on which treatment was given.

FIG. 5 shows water fall plots on day 16. DOHH2 tumor-bearing mice were treated with Antibody A2, bendamustine or with the combination of Antibody A2 and bendamustine. Individual changes from baseline at day 16 are plotted.

FIG. 6 shows the change of body weight over time. DOHH2 tumor-bearing mice were treated with Antibody A2, bendamustine or with the combination of Antibody A2 and bendamustine. Average changes of body weight are plotted over time. Day 1 was the first day, day 16 the last day of the experiment. The symbols on the top denote the days on which treatment was given.

### LEGEND TO SEQUENCE LISTING

SEQ ID NO 1: nucleic acid sequence variable heavy (Vh) chain

SEQ ID NO 2: amino acid sequence variable heavy chain

SEQ ID NO 3: nucleic acid sequence variable light (Vl) chain

SEQ ID NO 4: amino acid sequence variable light chain

SEQ ID NO 5: A2 heavy chain amino acid sequence

SEQ ID NO 6: A2 light chain amino acid sequence

SEQ ID NO 7: constant heavy chain amino acid sequence

SEQ ID NO 8: constant light chain amino acid sequence

SEQ ID NO 9: A4 heavy chain amino acid sequence

SEQ ID NO 10: A4 light chain amino acid sequence

SEQ ID NO 11: B2 heavy chain amino acid sequence

SEQ ID NO 12: B2 light chain amino acid sequence

SEQ ID NO 13: B4 heavy chain amino acid sequence

SEQ ID NO 14: B4 light chain amino acid sequence

SEQ ID NO 15: CDR1 heavy chain (H1)

SEQ ID NO 16: CDR2 heavy chain (H2)

SEQ ID NO 17: CDR3 heavy chain (H3)

SEQ ID NO 18: CDR1 light chain (L1)

SEQ ID NO 19: CDR2 light chain (L2)

SEQ ID NO 20: CDR3 light chain (L3)

SEQ ID NO 21: alternative CDR2 heavy chain (H2b)

### DETAILED DESCRIPTION OF THE INVENTION

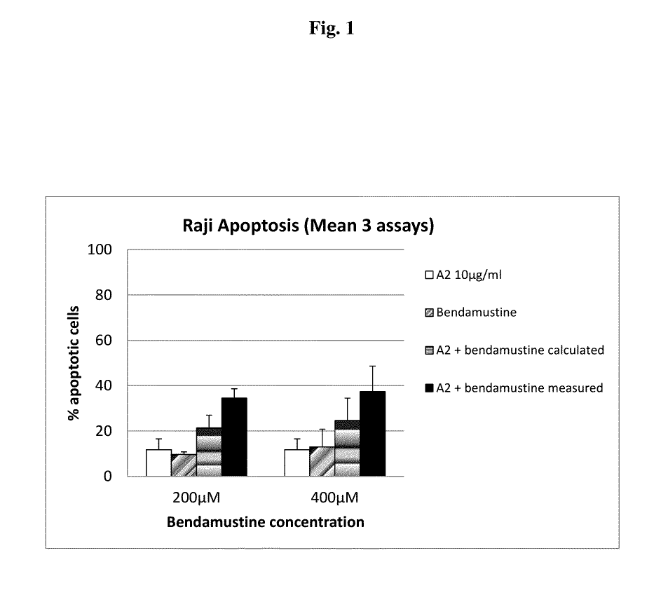

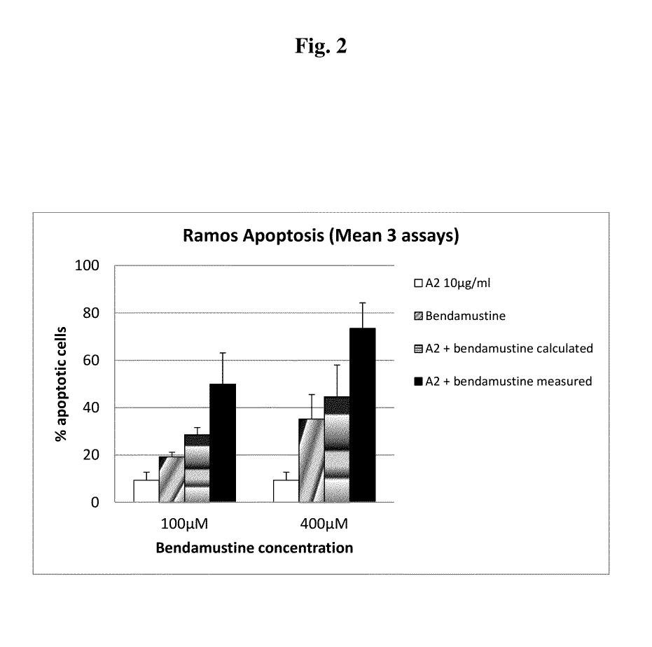

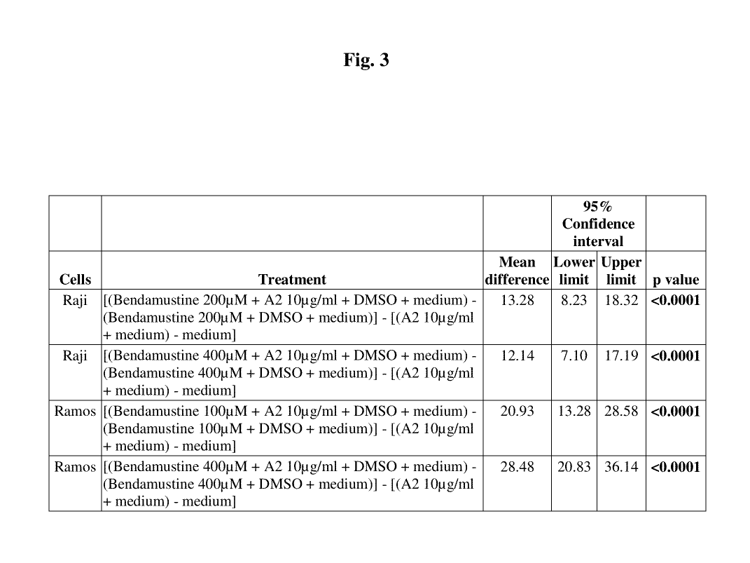

The antibody A2 (=mAb A2) is a potent inducer of apoptosis both in the absence and presence of an IgG cross-linking antibody (see patent application WO2009/019312). We investigated the ability of mAb A2 to induce apoptosis on CD37-positive lymphoma cell lines Ramos and Raji in the presence of the alkylating agent bendamustine in vitro. Ramos and Raji lymphoma cells were incubated for 48 hrs with mAb A2 at a concentration of 10 μg/ml, bendamustine at concentrations of 100 μM, 200 μM and 400 μM, or combinations thereof. Three independent experiments were performed for each cell line. The mean apoptosis induction is shown in FIG. 1 and FIG. 2. Mab A2 alone induced apoptosis in 12% of Raji cells and 9% of Ramos cells, respectively. Single agent bendamustine caused 10% (200 μM) and 13% (400 μM) apoptosis on Raji cells and 19% (100 μM) and 35% (400 μM) apoptosis on Ramos cells. The combination of mAb A2 with bendamustine induced significantly greater apoptosis than treatment with single agents. On Raji cells, the combination of mAb A2 with 200 μM bendamustine resulted in 35% apoptotic cells, the combination of mAb A2 with 400 μM bendamustine resulted in 37% apoptotic cells. On Ramos cells, the combination of mAb A2 with 100 μM bendamustine resulted in 50% apoptotic cells, the combination of mAb A2 with 400 μM bendamustine resulted in 73% apoptotic cells. The pro-apoptotic effect of the combination was surprisingly higher than the calculated additive effect of both individual treatments (FIGS. 1 and 2). Statistical analysis of interaction contrasts showed significant differences between the single agents and the combination groups, indicating synergistic activity of mAb A2 in combination with bendamustine (FIG. 3).

We also investigated the efficacy of antibody A2 in combination with bendamustine chemotherapy in a model of human follicular lymphoma (DOHH2) in C.B-17 scid mice. This data is presented and discussed in Example 3.

Antibody A2 and bendamustine were administered twice weekly intraperitoneally. Tumors were established from cultured DOHH2 cells by subcutaneous injection. Tumor volumes were determined three times a week using a caliper. Body weight of the mice was measured as an indicator of tolerability of the compounds on the same days. Day 1 was the first, day 16 the last day of the study. It was determined that a combination of antibody A2 and bendamustine was significantly more efficacious than the single agent treatment with antibody A2 or with bendamustine. All 7 tumors each treated with either antibody A2 or bendamustine were growth inhibited while 6 out of 7 tumors treated with the combination completely regressed and one out of 7 partially regressed to a volume of only 9 mm3. Importantly statistical analysis showed synergism of the combination treatment. Hence there is a synergistic activity of mAb A2 in combination with bendamustine in vivo.

An important finding from the studies reported in this patent application is the fact that mAb A2 exerts its pro-apoptotic activity without the need of an IgG cross-linking antibody, both as single agent and in combination with bendamustine. IgG cross-linking in vitro is thought to mimic cross-linking by immune effector cells, e.g. NK cells, in vivo. Several antibodies described in the literature are dependent on IgG cross-linking to induce apoptosis, in particular the CD37-targeting antibody-like molecule CAS024 depends on IgG cross-linking (see European patent EP 2 132 228 B1). In cancer patients in vivo, the presence of immune effector cells may be limited or reduced, especially in patients treated with chemotherapeutic agents. Hence, an antibody which is able to induce apoptosis in the absence of an IgG cross-linking agent is considered favorable compared to an antibody which depends on IgG cross-linking, especially in combination with a chemotherapeutic agent which potentially impairs immune effector cell activity. A2 is such an antibody which in combination with bendamustine is able to induce surprisingly more than additive apoptosis than either agent alone without the need for IgG cross-linking, which is considered advantageous for the treatment of cancer patients.

In an in vivo CB-17 SCID mouse model the combination of a CD37 antibody (such as mAb A2 or B2, preferably mAb A2) and bendamustine shows an improved anti-tumor effect over that of single agent treatment. These results show that a combination of a CD37 antibody (such as mAb A2 or B2, preferably mAb A2) with bendamustine results in superior anti-tumor efficacy compared to single agent treatment.

Definitions

Terms not specifically defined herein should be given the meanings that would be given to them by one of skill in the art in light of the disclosure and the context. The general embodiments “comprising” or “comprised” encompass the more specific embodiment “consisting of”. Furthermore, singular and plural forms are not used in a limiting way. As used in the specification, however, unless specified to the contrary, the following terms have the meaning indicated and the following conventions are adhered to.

The term “bendamustine” or (more specifically) “bendamustine hydrochloride” describes a chemotherapeutic agent. Bendamustine (INN, trade names Ribomustin and Treanda; also known as SDX-105) is a nitrogen mustard used in the treatment of hematologic malignancies, e.g. chronic lymphocytic leukemias and lymphomas. It belongs to the family of drugs called alkylating agents, which are widely used for the treatment of malignant neoplasms (cancer). The chemical mass formula is C₁₆H₂₁Cl₂N₃O₂ with a molecular mass of 358.262 g/mol. The systematic (IUPAC) name is 4-[5-[Bis(2-chloroethyl)amino]-1-methylbenzimidazol-2-yl]butanoic acid. The chemical structure of bendamustine is as follows:

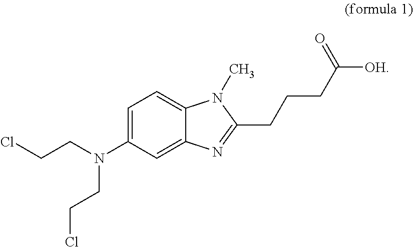 [SMILES: Cn1c(CCCC(=O)O)nc2cc(N(CCCl)CCCl)ccc21]

“CD37”, a member of the tetraspanin superfamily, is a heavily glycosylated cell surface molecule with four transmembrane domains and two extracellular loops. CD37 is predominantly expressed on B-cells and B-cell malignancies, low level expression of CD37 has been reported on T-cells, granulocytes, and monocytes. High levels of CD37 expression have been observed in samples of patients with chronic lymphocytic leukemia (CLL) and different subtypes of non-Hodgkin's lymphoma (NHL) including mantle cell lymphoma (MCL) (Schwartz-Albiez et al, Journal Immunol 140: 905-914, 1988; Barrena et al., Leukemia 19: 1376-1383, 2005). This expression pattern makes CD37 an attractive target for antibody-mediated cancer therapy. Binding of a CD37-specific mAb to cancer cells may trigger various mechanisms of action: First, after the antibody binds to the extracellular domain of the CD37 antigen, it may activate the complement cascade and lyse the targeted cell. Second, an anti-CD37 antibody may mediate antibody-dependent cell-mediated cytotoxicity (ADCC) to the target cell, which occurs after the Fc portion of the bound antibody is recognized by appropriate receptors on cytotoxic cells of the immune system. Third, the antibody may alter the ability of B-cells to respond to antigen or other stimuli. Finally, anti-CD37 antibody may initiate programmed cell death (apoptosis).

“CD37 positive”, “CD37 positive cells” or “CD37 positive malignancies” means that the detection of CD37 is possible/feasible by immunohistochemistry, flow cytometry such as FACS (fluorescence activated cell sorter) analysis (of e.g. blood, bone marrow or cell suspensions) or alternative techniques. Suitable assays to detect CD37 positive cells/malignancies are well known to a person skilled in the art.

The terms “CD37 antibody”, “CD37 antibody molecule”, “anti-CD37 antibody” and “anti-CD37 antibody molecule” as used in the present invention specifically relate to an antibody with a binding specificity for CD37 antigen. Examples of such antibodies are known in the art and are further described below.

The terms “anti-CD37 antibody molecule”, “anti-CD37 antibody”, “CD37 antibody” and “CD37 antibody molecule” are used interchangeably.

The term “CD37 antibody” or “anti-CD37 antibody molecule” encompasses anti-CD37 antibodies and anti-CD37 antibody fragments as well as conjugates with antibody molecules. Antibodies include, in the meaning of the present invention, chimeric monoclonal and humanized monoclonal antibodies. The term “antibody”, which may interchangeably be used with “antibody molecule”, shall encompass complete immunoglobulins (as they are produced by lymphocytes and for example present in blood sera), monoclonal antibodies secreted by hybridoma cell lines, polypeptides produced by recombinant expression in host cells, which have the binding specificity of immunoglobulins or monoclonal antibodies, and molecules which have been derived from such antibodies by modification or further processing while retaining their binding specificity.

In certain embodiments, the antibody molecule of the invention is a chimeric CD37-specific antibody that has the heavy chain variable region of a non-human antibody defined in a) or b) fused to the human heavy chain constant region IgG1 and the light chain variable region of a non-human antibody defined in a) or b) fused to the human light chain constant region kappa.

The CD37 antibody may also be in the form of a conjugate, i.e. an antibody molecule that is chemically coupled to a cytotoxic agent, particularly a cytotoxic agent that induces cytotoxicity (e.g. apoptosis or mitotic arrest) of tumor cells. As a result of normal pharmacologic clearance mechanisms, an antibody employed in a drug conjugate (an “immunoconjugate”) contacts and binds to target cells only in limited amounts. Therefore, the cytotoxic agent employed in the conjugate must be highly cytotoxic such that sufficient cell killing occurs to elicit a therapeutic effect. As described in US 2004/0241174, examples of such cytotoxic agents include taxanes (see, e.g. WO 01/38318 and WO 03/097625), DNA-alkylating agents (e.g., CC-1065 analogs), anthracyclines, tubulysin analogs, duocarmycin analogs, doxorubicin, auristatin E, ricin A toxin, and cytotoxic agents comprising a reactive polyethylene glycol moiety (see, e.g., Sasse et al., 2000; Suzawa et al., 2000; Ichimura et al., 1991; Francisco et al., 2003; U.S. Pat. No. 5,475,092; U.S. Pat. No. 6,340,701; U.S. Pat. No. 6,372,738; and U.S. Pat. No. 6,436,931; US 2001/0036923; US 2004/0001838; US 2003/0199519; and WO 01/49698).

In a preferred embodiment, the cytotoxic agent is a maytansinoid, i.e. a derivative of maytansine (CAS 35846538), maytansinoids being known in the art to include maytansine, maytansinol, C-3 esters of maytansinol, and other maytansinol analogues and derivatives (see, e.g., U.S. Pat. No. 5,208,020; and U.S. Pat. No. 6,441,163).

Anti-CD37 antibody immunoconjugates may be designed and synthesized as described in WO 2007/077173 for anti-FAP immunoconjugates.

In a further embodiment, the anti-CD37 molecule of the invention may be radioactively labelled to form a radioimmunoconjugate, an approach suggested for the anti-CD37 antibody MB-1 (Buchsbaum et al., 1992, see above). Radionuclides with advantageous radiation properties are known in the art, examples are Phosphorus-32, Strontium-89, Yttrium-90, Iodine-131, Samarium-153, Erbium-169, Ytterbium-175, Rhenium-188, that have been successfully and stably coupled to MAbs. The CD37 antibodies of the invention may be labelled with various radionuclides using direct labelling or indirect labelling methods known in the art, as described in U.S. Pat. No. 6,241,961. A review on technologies for generating and applying novel radiolabled antibody conjugates that are useful in the present invention, is given by Goldenberg and Sharkey, 2007.

An antibody molecule of the invention, whether Fc-engineered or not, may also be bispecific, i.e. an antibody molecule that binds to two different targets, one of them being CD37, the other one being selected from e.g. surface antigens expressed by T cells, e.g. CD3, CD16 and CD56.

The term “antibody” or “antibodies” comprises monoclonal, polyclonal, multispecific and single chain antibodies and fragments thereof such as for example Fab, Fab′, F(ab′)₂, Fc and Fc′ fragments, light (L) and heavy (H) immunoglobulin chains and the constant, variable or hypervariable regions thereof as well as Fv and Fd fragments. The term “antibody” or “antibodies” comprises antibodies of human or non-human origin, humanised as well as chimeric antibodies and furthermore Fc-engineered antibodies or Fc-fusion molecules.

Fab fragments (fragment antigen binding=Fab) consist of the variable regions of both chains which are held together by the adjacent constant regions. They may be produced for example from conventional antibodies by treating with a protease such as papain or by DNA cloning. Other antibody fragments are F(ab′)₂ fragments which can be produced by proteolytic digestion with pepsin.

By gene cloning it is also possible to prepare shortened antibody fragments which consist only of the variable regions of the heavy (VH) and light chain (VL). These are known as Fv fragments (fragment variable=fragment of the variable part). As covalent binding via the cysteine groups of the constant chains is not possible in these Fv fragments, they are often stabilised by some other method. For this purpose the variable regions of the heavy and light chains are often joined together by means of a short peptide fragment of about 10 to 30 amino acids, preferably 15 amino acids. This produces a single polypeptide chain in which VH and VL are joined together by a peptide linker. Such antibody fragments are also referred to as single chain Fv fragments (scFv). Examples of scFv antibodies are known in the art.

In past years various strategies have been developed for producing multimeric scFv derivatives. The intention is to produce recombinant antibodies with improved pharmacokinetic properties and increased binding avidity. In order to achieve the multimerisation of the scFv fragments they are produced as fusion proteins with multimerisation domains. The multimerisation domains may be, for example, the CH3 region of an IgG or helix structures (“coiled coil structures”) such as the Leucine Zipper domains. In other strategies the interactions between the VH and VL regions of the scFv fragment are used for multimerisation (e.g. dia, tri- and pentabodies).

The term “diabody” is used in the art to denote a bivalent homodimeric scFv derivative. Shortening the peptide linker in the scFv molecule to 5 to 10 amino acids results in the formation of homodimers by superimposing VH/VL chains. The diabodies may additionally be stabilised by inserted disulphite bridges. Examples of diabodies can be found in the literature.

The term “minibody” is used in the art to denote a bivalent homodimeric scFv derivative. It consists of a fusion protein which contains the CH3 region of an immunoglobulin, preferably IgG, most preferably IgG1, as dimerisation region. This connects the scFv fragments by means of a hinge region, also of IgG, and a linker region. Examples of such minibodies are known in the art.

The term “triabody” is used in the art to denote a trivalent homotrimeric scFv derivative. The direct fusion of VH-VL without the use of a linker sequence leads to the formation of trimers.

The fragments known in the art as mini antibodies which have a bi, tri- or tetravalent structure are also derivatives of scFv fragments. The multimerisation is achieved by means of di-, tri- or tetrameric coiled coil structures.

There are also “scaffold proteins” or “scaffold antibodies” known in the art. Using this term, a scaffold protein means any functional domain of a protein, especially an antibody, that is coupled by genetic cloning or by co-translational processes with another protein or part of a protein that has another function.

The term “Complementary determining region” or “CDR” or “CDRs” of an antibody/antibody molecule means the hypervariable regions (also called Complementarity Determining Regions, abbreviated to “CDRs”) of immunoglobulins. The CDRs were originally defined by Kabat et al., (“Sequences of Proteins of Immunological Interest” Kabat, E., of al., U.S. Department of Health and Human Services, (1983) and Kabat E. A., Wu T. T., Perry H. M., Gottesman K. S. and Foeller C. Sequences of Proteins of Immunological Interest (5th Ed.). NIH Publication No. 91-3242. U.S. Department of Health and Human Services, Public Health Service, National Institutes of Health, Bethesda, Md. 1991) based on extent of sequence variability of numerous antibody sequences. The CDRs are believed to contact the target antigen of an antibody and to be primarily responsible for binding. Chothia et al (Chothia and Lesk, J. Mol. Biol., 196:901-917 (1987)) have given an alternate definition of the hypervariable regions or CDRs. The Chothia definition is based on the residues that constitute the loops in the 3-dimensional structures of antibodies.

In the specific context of the present invention the CDRs are determined on the basis of the Kabat system. From the sequences of the variable regions as shown in SEQ ID NO:2 and SEQ ID NO:4, the CDR sequence can be routinely determined by searching the Kabat sequence database for sequence features. The 3 CDRs contained within the variable heavy chain as shown in SEQ ID NO:2 comprise preferably positions 31-35 (H1, SEQ ID NO: 15), 50-66 (H2, SEQ ID NO: 16) or 50-62 (H2b, SEQ ID NO: 21) and 99-105 (H3, SEQ ID NO: 17), the 3 CDRs contained within the variable light chain as shown in SEQ ID NO:4 comprise preferably positions 24-34 (L1, SEQ ID NO: 18), 50-56 (L2, SEQ ID NO: 19) and 89-97 (L3, SEQ ID NO: 20).

The term “treatment cycle” describes a time period of between 1 to 6 weeks, preferably 3 to 4 weeks, most preferably 4 weeks, wherein the patient receives at least one dose of the CD37 antibody and at least one dose of bendamustine.

The terms “dose” and “dosage” are used interchangeably.

The terms “NHL” and “B-NHL” are used interchangeably.

### Embodiments

The present invention concerns a CD37 antibody for use in a method for the treatment of a patient suffering from a CD37-positive malignancy, preferably a B-cell malignancy, most preferably chronic lymphocytic leukemia (CLL) or B-cell non-Hodgkin's lymphoma (B-NHL), in combination with bendamustine, whereby the CD37 antibody comprises:
a variable heavy chain comprising CDRs have the SEQ ID NOs: 15, 16 or 21, and 17, and
a variable light chain comprising CDRs having the SEQ ID NOs: 18, 19 and 20.

In a specific embodiment the CD37 antibody is a chimeric antibody. Preferably said chimeric antibody comprises the human constant heavy chain amino acid sequence SEQ ID NO:7 and the human constant light chain amino acid sequence SEQ ID NO:8.

In a preferred embodiment the CD37 antibody comprises the heavy chain amino acid sequence SEQ ID NO:5 and the light chain amino acid sequence SEQ ID NO:6 (=>A2).

In a specific embodiment the CD37 antibody is a humanized antibody. Preferably said humanized CD37 antibody comprises the heavy chain amino acid sequence SEQ ID NO: 11 and the light chain amino acid sequence 12 (=>B2).

In a specific embodiment the patient receives at least one dose of the CD37 antibody and at least one dose of bendamustine during a treatment cycle, whereby a treatment cycle is a time period of about 1 to 6 weeks, preferably 3 to 4 weeks, most preferably 4 weeks.

In a further specific embodiment the CD37 antibody is administered to said patient simultaneously with the administration of bendamustine.

In another embodiment the CD37 antibody is administered to said patient after the administration of bendamustine, preferably within 24 hrs or within 36 hrs after the administration of bendamustine.

In a further embodiment the CD37 antibody is administered to said patient before the administration of bendamustine, preferably within 24 hrs or within 36 hrs before the administration of bendamustine.

In another preferred embodiment the CD37 antibody is administered to said patient after a 2 day consecutive application of bendamustine, preferably within 24 hrs or within 36 hrs after the administration of the second bendamustine dosage. In another preferred embodiment the CD37 antibody is administered to said patient the day after a 2 day consecutive application of bendamustine, whereby the day after preferably means within 24 hrs or within 36 hrs after the administration of bendamustine. Preferably bendamustine is administered to said patient on days 1 and 2 of a 1 to 6 week treatment cycle, more preferably of a 3-4 week treatment cycle, most preferably of a 4 week treatment cycle, and the CD37 antibody is administered on day 3 of the treatment cycle.

In a further preferred embodiment the CD37 antibody is administered to said patient before a 2 day consecutive application of bendamustine, preferably within 24 hrs or within 36 hrs before the administration of the first bendamustine dosage. In another preferred embodiment the CD37 antibody is administered to said patient the day before a 2 day consecutive application of bendamustine, whereby the day before preferably means within 24 hrs or within 36 hrs before the administration of bendamustine. Preferably bendamustine is administered to said patient on days 2 and 3 of a 1 to 6 week treatment cycle, more preferably of a 3-4 week treatment cycle, most preferably of a 4 week treatment cycle, and the CD37 antibody is administered on day 1 of the treatment cycle.

In a specific embodiment the CD37 antibody is additionally administered at least one more time in between, preferably in the middle of the treatment cycle at about 2 weeks. In another embodiment the CD37 antibody is additionally administered at least one more time during a treatment cycle, preferably in the middle of the treatment cycle at about 2 weeks or once weekly, whereby the treatment cycle is a time period of between 1 to 6 weeks, preferably 3 to 4 weeks, most preferably 4 weeks. The treatment cycle is a time period of between 1 to 6 weeks, preferably 3 to 4 weeks, most preferably 4 weeks, wherein the patient receives at least one dose of the CD37 antibody and at least one dose of bendamustine.

The CD37 antibody, preferably A2 (CD37 antibody comprising SEQ ID Nos:5 and 6) and B2 (CD37 antibody comprising SEQ ID Nos:11 and 12), most preferably A2, is administered in a dose of about 0.01 μg/kg to 40 mg/kg. Administration to the patient may occur by one or more separate administrations. It may occur for example by infusion such as continuous infusion.

For A2 (CD37 antibody comprising SEQ ID Nos:5 and 6), the estimated weekly dose for a 70 kg human is in the range of 1 mg to 2800 mg, preferably 1 mg to 400 mg weekly or 2 mg to 800 mg every 2 weeks. The estimated human weekly dose of B2 (CD37 antibody comprising SEQ ID Nos:11 and 12) for a 70 kg human is in the range of 1 mg to 2800 mg, preferably in the range of 1 mg to 1000 mg, e.g. 100 mg to 385 mg weekly or 200 mg to 770 mg every two weeks for a 70 kg person.

Bendamustine is preferably dosed on two consecutive days (e.g. d1+d2) of a treatment cycle, which is preferably 3-4 weeks (=21-28 days) long. The dose for bendamustine ranges between 50-150 mg/m2 body surface on 2 treatment days of a 3 to 4 week long treatment cycle.

Preferably the dose of bendamustine ranges between 70-120 mg/m² body surface or between 100-150 mg/m² body surface on d1+d2 of a treatment cycle.

For the treatment of a CLL patient bendamustine is preferably administered at a dosage of 100 mg/m² body surface on days 1 and 2 of the treatment cycle, which is preferably 3-4 weeks long, most preferably 4 weeks.

For the treatment of a NHL patient bendamustine is preferably administered at a dosage of 120 mg/m² body surface on days 1 and 2 of the treatment cycle, which is preferably 3-4 weeks long, most preferably 3 weeks.

Furthermore preferred is a bendamustine dose in the range of 60-70 mg/m² body surface on d1+d2 of a treatment cycle.

In a further specific embodiment bendamustine is administered as a one-time administration per treatment cycle preferably with a dose of 70-400 mg/m² body surface.

The bendamustine dose as described above is administered preferably on day 1 and on day 2 of a 3-4 week treatment cycle. Furthermore, preferred is the administration of a bendamustine dose as described above on 2 consecutive days following a CD37 antibody administration (e.g. day 1=CD37 administration in any of the dosages as described above, days 2+3=bendamustine administration in any of the dosages as described above) of a preferably 3-4 week long treatment cycle.

The bendamustine dose may be administered by any way, e.g. infusion, parenteral or oral administration. Preferably the dose range for oral administration of bendamustine ranges from 10 to 1000 mg, more preferably 25 to 600 mg or 50 to 200 mg, most preferably about 100 mg.

The CD37 antibody dose may be administered by any way, e.g. infusion such as continuous infusion, subcutaneous injection, inhalation, parenteral or oral administration.

In a specific embodiment of the present in invention a CD37 antibody is administered in combination with bendamustine as first line treatment. First line treatment means as a first treatment option (before other treatment options are performed/used). In a preferred embodiment of the present in invention a CD37 antibody is administered in combination with bendamustine as second line treatment of CLL.

In another specific embodiment of the present in invention a CD37 antibody is administered in combination with bendamustine as second line or third or fourth or further line treatment.

Second, third, fourth or further line treatment means the administration as a second, third, fourth or later/further line treatment option after one or more other treatment(s) already has (have) been performed/used.

For the treatment of a patient suffering from CLL a preferred treatment cycle scheme lasts for a time period of 4 weeks, whereby bendamustine is preferably administered at a dose of 100 mg/m² body surface preferably on day 1 and 2 and whereby at least one CD37 antibody, preferably A2 or B2, is administered at a dose as described above either before, after or simultaneously with the bendamustine administration. Simultaneously hereby means on the same day. Simultaneously furthermore may mean within six hours of each other or within one hour of each other or with the same injection. Furthermore, another preferred treatment cycle scheme for CLL comprises additional administration(s) of CD37 antibody in between, for example in the middle of the treatment cycle at about 2 weeks.

For the treatment of a patient suffering from NHL a preferred treatment cycle scheme lasts for a time period of 3 weeks, whereby bendamustine is preferably administered at a dose of 120 mg/m2 body surface preferably on day 1 and 2 and whereby at least one CD37 antibody, preferably A2 or B2, is administered at a dose as described above either before, after or simultaneously with the bendamustine administration. Simultaneously hereby means on the same day. Simultaneously furthermore may mean within six hours of each other or within one hour of each other or with the same injection. Furthermore, another preferred treatment cycle scheme for NHL comprises additional administration(s) of CD37 antibody in between, for example once a week, thus resulting in several, preferably 3 to 4, most preferably 4 administrations of CD37 antibody per treatment cycle.
The present invention further concerns a method of reducing CD37-positive cells, more specifically B-cells, comprising exposing B-cells to a combination of a CD37 antibody and bendamustine, whereby said CD37 antibody comprises:
a) a variable heavy chain comprising CDRs have the SEQ ID NOs: 15, 16 or 21, and 17, and
b) a variable light chain comprising CDRs having the SEQ ID NOs: 18, 19 and 20.
The present invention furthermore concerns a method of depleting CD37 expressing B-cells from a population of cells comprising administering to said population of cells: a) a CD37 antibody or a pharmaceutical composition comprising a CD37 antibody and b) bendamustine, wherein said method is preferably carried out in vitro, and whereby said CD37 antibody comprises:
a) a variable heavy chain comprising CDRs have the SEQ ID NOs: 15, 16 or 21, and 17, and
b) a variable light chain comprising CDRs having the SEQ ID NOs: 18, 19 and 20.
The present invention further concerns a method of reducing CD37-positive cells comprising:
Exposing CD37-positive cells to a CD37 antibody and
Exposing CD37-positive cells to bendamustine,
whereby said CD37 antibody of step a) comprises:
i) a variable heavy chain comprising CDRs have the SEQ ID NOs: 15, 16 or 21, and 17, and
ii) a variable light chain comprising CDRs having the SEQ ID NOs: 18, 19 and 20.
The present invention furthermore concerns a method of reducing B-cells comprising:
Exposing B-cells to a CD37 antibody and
Exposing B-cells to bendamustine,
whereby said CD37 antibody of step a) comprises:
i) a variable heavy chain comprising CDRs have the SEQ ID NOs: 15, 16 or 21, and 17, and
ii) a variable light chain comprising CDRs having the SEQ ID NOs: 18, 19 and 20.

In a specific embodiment the CD37 antibody is a chimeric antibody. Preferably said chimeric antibody comprises the human constant heavy chain amino acid sequence SEQ ID NO:7 and the human constant light chain amino acid sequence SEQ ID NO:8.

In a preferred embodiment the CD37 antibody comprises the heavy chain amino acid sequence SEQ ID NO:5 and the light chain amino acid sequence SEQ ID NO:6 (=>A2).

In a specific embodiment the CD37 antibody is a humanized antibody. Preferably said humanized CD37 antibody comprises the heavy chain amino acid sequence SEQ ID NO: 11 and the light chain amino acid sequence 12 (=>B2).

In a specific embodiment of any of said methods the CD37-positive cells are exposed to the CD37 antibody and bendamustine simultaneously. Said CD37-positive cells are preferably B-cells.

In another embodiment of any of said methods the CD37-positive cells are exposed to the CD37 antibody after they are exposed to bendamustine, preferably within 24 hrs or within 36 hrs after they are exposed to bendamustine. Said CD37-positive cells are preferably B-cells.

In a further embodiment of any of said methods the CD37-positive cells are exposed to the CD37 antibody before they are exposed to bendamustine, preferably within 24 hrs or within 36 hrs before they are exposed to bendamustine. Said CD37-positive cells are preferably B-cells.

In a specific embodiment said method is carried out in vivo.

In a specific embodiment said method is carried out in vitro.

The present invention further concerns a kit for reducing CD37-positive cells comprising:

a container comprising a CD37 antibody, whereby said CD37 antibody comprises:

a variable heavy chain comprising CDRs having the SEQ ID NOs: 15, 16 or 21, and 17, and

a variable light chain comprising CDRs having the SEQ ID NOs: 18, 19 and 20, and

b) a protocol for using the kit to reduce CD37-positive cells in combination with bendamustine. Said CD37-positive cells are preferably B-cells.

The present invention furthermore concerns a kit for reducing CD37-positive cells comprising:

a first container comprising a CD37 antibody, whereby said CD37 antibody comprises:

a variable heavy chain comprising CDRs have the SEQ ID NOs: 15, 16 or 21, and 17, and

a variable light chain comprising CDRs having the SEQ ID NOs: 18, 19 and 20, and

a second container comprising bendamustine, and

c) a protocol for using the kit to reduce CD37-positive cells. Said CD37-positive cells are preferably B-cells.

In a specific embodiment the protocol in step c) indicates to administer the CD37 antibody and bendamustine simultaneously.

In another embodiment the protocol in step c) indicates to administer the CD37 antibody before bendamustine, preferably within 24 hrs or within 36 hrs before the administration of bendamustine.

In a further embodiment the protocol in step c) indicates to administer the CD37 antibody after bendamustine, preferably within 24 hrs or within 36 hrs after the administration of bendamustine.

In a specific embodiment the protocol in step c) indicates to administer the kit components to a patient suffering from a CD37-positive malignancy, preferably a B-cell malignancy, preferably chronic lymphocytic leukemia (CLL) or NHL, most preferably CLL.

In a further specific embodiment the protocol in step c) indicates that the patient receives at least one dose of the CD37 antibody and at least one dose of bendamustine during a treatment cycle, whereby a treatment cycle is a time period of about 1 to 6 weeks, preferably 3 to 4 weeks, most preferably 4 weeks.

In further specific embodiments the protocol in step c) indicates treatment cycles and/or dosage schemes as described above for the second medical use of the described CD37 antibodies.

The present invention further concerns an article of manufacture comprising a CD37 antibody and bendamustine and a label indicating a method as described above, whereby the CD37 antibody comprises: a) a variable heavy chain comprising CDRs have the SEQ ID NOs: 15, 16 or 21, and 17, and b) a variable light chain comprising CDRs having the SEQ ID NOs: 18, 19 and 20.

The present invention furthermore concerns a pharmaceutical composition comprising, a CD37 antibody, bendamustine, and a pharmaceutically acceptable carrier, whereby the CD37 antibody comprises:

a) a variable heavy chain comprising CDRs have the SEQ ID NOs: 15, 16 or 21, and 17, and

b) a variable light chain comprising CDRs having the SEQ ID NOs: 18, 19 and 20.

In a specific embodiment the pharmaceutical composition comprises as the active ingredient a CD37 antibody and bendamustine, and additionally a pharmaceutically acceptable carrier, whereby the CD37 antibody comprises:

a) The CDRs contained within the variable heavy chain as shown in SEQ ID NO:2, preferably said CDRs have SEQ ID NOs: 15, 16 or 21, and 17, and

b) The CDRs contained within the variable light chain as shown in SEQ ID NO:4, preferably said CDRs have SEQ ID NOs: 18, 19 and 20.

The present invention further concerns the pharmaceutical composition as described above for use as a medicament.

The present invention furthermore concerns the pharmaceutical composition as described above for use in a method for the treatment of a patient suffering from a B-cell malignancy, preferably for use in a method for the treatment of a chronic lymphocytic leukemia (CLL) patient.

The present invention further concerns a method of treating a B-cell malignancy comprising administering a therapeutically effective amount of a CD37 antibody in combination with bendamustine to a patient in need thereof, whereby the CD37 antibody comprises:

a) a variable heavy chain comprising CDRs have the SEQ ID NOs: 15, 16 or 21, and 17, and

b) a variable light chain comprising CDRs having the SEQ ID NOs: 18, 19 and 20.

The present invention furthermore concerns a method for treating a patient suffering from a B-cell malignancy selected from B-cell non-Hodgkins lymphoma, B-cell chronic lymphocytic leukemia and multiple myeloma, comprising administering to said patient an effective amount of a pharmaceutical composition of the present invention.

The present invention further concerns a method of treating a B-cell malignancy comprising administrating a therapeutically effective amount of a) A CD37 antibody and b) Bendamustine, to a patient in need thereof, whereby the CD37 antibody comprises:

a) a variable heavy chain comprising CDRs have the SEQ ID NOs: 15, 16 or 21, and 17, and

b) a variable light chain comprising CDRs having the SEQ ID NOs: 18, 19 and 20.

In a specific embodiment of said methods of treatment the patient receives at least one dose of the CD37 antibody and at least one dose of bendamustine during a treatment cycle, whereby a treatment cycle is a time period of about 1 to 6 weeks, preferably 3 to 4 weeks, most preferably 4 weeks.
In a specific embodiment of any of said methods the B-cells are exposed to the CD37 antibody and bendamustine simultaneously.
In another embodiment of any of said methods the B-cells are exposed to the CD37 antibody after they are exposed to bendamustine, preferably within 24 hrs or within 36 hrs after they are exposed to bendamustine.
In a further embodiment of any of said methods the B-cells are exposed to the CD37 antibody before they are exposed to bendamustine, preferably within 24 hrs or within 36 hrs before they are exposed to bendamustine.
In a specific embodiment said method is carried out in vivo.
In a specific embodiment said method is carried out in vitro.
The dosage regimens described above for the second medical use of CD37 antibodies in combination with bendamustine likewise apply for the described methods of treatment of the present invention.
The present invention further concerns the CD37 antibody as described, any of the methods as described, the kit as described, the article of manufacture as described, the pharmaceutical composition as described, and the methods of treatment as described, whereby the CD37 antibody is a chimeric antibody defined by
a) a variable heavy chain comprising the amino acid sequence shown in SEQ ID NO: 2, and
b) a variable light chain comprising the amino acid sequence shown in SEQ ID NO:4,

whereby the constant heavy and light chains are preferably of human origin.

The present invention furthermore concerns the CD37 antibody as described, any of the methods as described, the kit as described, the article of manufacture as described, the pharmaceutical composition as described, and the methods of treatment as described, whereby the antibody has a heavy chain comprising the amino acid sequence of SEQ ID NO:5 and a light chain comprising the amino acid sequence of SEQ ID NO:6.

The present invention furthermore concerns the CD37 antibody as described, any of the methods as described, the kit as described, the article of manufacture as described, the pharmaceutical composition as described, and the methods of treatment as described, the antibody has a heavy chain comprising the amino acid sequence of SEQ ID NO: 7 fused to SEQ ID NO:2 and a light chain comprising the amino acid sequence of SEQ ID NO: 8 fused to SEQ ID NO:4.

The present invention furthermore concerns the CD37 antibody as described, any of the methods as described, the kit as described, the article of manufacture as described, the pharmaceutical composition as described, and the methods of treatment as described, whereby the antibody has a heavy chain comprising the amino acid sequence of SEQ ID NO:9 and a light chain comprising the amino acid sequence of SEQ ID NO:10.

The present invention furthermore concerns the CD37 antibody as described, any of the methods as described, the kit as described, the article of manufacture as described, the pharmaceutical composition as described, and the methods of treatment as described, whereby said antibody is a humanized antibody defined by frameworks supporting said CDRs that are derived from a human antibody, and wherein the constant heavy and light chains are from a human antibody.

The present invention furthermore concerns the CD37 antibody as described, any of the methods as described, the kit as described, the article of manufacture as described, the pharmaceutical composition as described, and the methods of treatment as described, whereby the antibody has a heavy chain comprising the amino acid sequence of SEQ ID NO:11 and a light chain comprising the amino acid sequence of SEQ ID NO:12.

The present invention furthermore concerns the CD37 antibody as described, any of the methods as described, the kit as described, the article of manufacture as described, the pharmaceutical composition as described, and the methods of treatment as described, whereby the antibody has a heavy chain comprising the amino acid sequence of SEQ ID NO:13 and a light chain comprising the amino acid sequence of SEQ ID NO:14.

The present invention furthermore concerns the CD37 antibody as described, any of the methods as described, the kit as described, the article of manufacture as described, the pharmaceutical composition as described, and the methods of treatment as described, whereby the CD37-positive malignancy is selected from the group consisting of: B-cell lymphomas, agressive B-cell lymphoma, Hodgkin's disease, B-cell non-Hodgkin's lymphoma (NHL), lymphomas, Waldenström's macroglobulinaemia (also called lymphoplasmacytic lymphoma or immunocytoma), central nervous system lymphomas, leukemias, acute lymphoblastic leukemia (ALL), chronic lymphocytic leukemia (CLL; also termed B-cell chronic lymphocytic leukemia BCLL), hairy cell leukemia, chronic myoblastic leukemia, myelomas, multiple myeloma, T-cell lymphoma, small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt's lymphoma/leukemia, grey zone lymphoma, B-cell proliferations of uncertain malignant potential, lymphomatoid granulomatosis, and post-transplant lymphoproliferative disorder, whereby the CD37-positive malignancy is preferably a B-cell malignancy, preferably B-cell non-Hodgkin's lymphoma, B-cell chronic lymphocytic leukemia, whereby the B-cell malignancy is most preferably chronic lymphocytic leukemia (CLL).

The practice of the present invention will employ, unless otherwise indicated, conventional techniques of medicine, pharmacy, chemistry, biology, oncology, cell biology, molecular biology, cell culture, immunology and the like which are in the skill of one in the art. These techniques are fully disclosed in the current literature.

The following examples are not limiting. They merely show possible embodiments of the invention. A person skilled in the art could easily adjust the conditions to apply it to other embodiments.

Experimental

Materials and Methods

Antibodies and Reagents

Antibody A2 was expressed in DHFR-deficient Chinese hamster ovary (CHO) DG44 suspension cells under serum-free conditions and purified via MabSelect Protein A affinity chromatography (GE Healthcare). The antibody was formulated in citrate buffer at a concentration of 10 mg/ml and stored between 4° and 8° C. Bendamustine (Ribomustine) was purchased from Mundipharma, Limburg, Germany. A stock solution of bendamustine (50 mM) was prepared in DMSO as disolvent. Aliquots of the stock solution were stored at −20° C. and diluted with cell culture medium to the final assay concentration immediately before use. Ramos (ATCC #CRL-1596) and Raji (ATCC #CCL-86) Burkitt lymphoma cells lines were cultured as recommended by the supplier.

Apoptosis Assay

Apoptosis was determined in Ramos and Raji Burkitt lymphoma cells after 48-hour incubation with antibody in the presence or absence of bendamustine by Annexin V and propidium iodide (PI) staining. For determination of apoptosis using Annexin V staining, 100 μL of cells, at a density of 1×10exp6 cells/mL in culture medium (RPMI 1640 with 10% FCS), were seeded into a 96-well round-bottom plate. 100 μL of antibody dilution, bendamustine and controls (in culture medium) were added to the cells. Incubation was performed at 37° C. in a humidified CO2 incubator for 48 hours. Thereafter, 100 μL supernatant was removed from each well. Staining for apoptotic cells was performed using the Vybrant™ Apoptosis Assay Kit 2 (Invitrogen #V13241). 5 μL Alexa Fluor® 488 Annexin V (Component A) and 1 μL propidium iodide (PI) (100 μg/mL PI stock 1:10 diluted with Annexin V binding buffer) were added to the cells and incubated for 15 minutes at room temperature in the dark. 150 μL ice-cold Annexin V binding buffer was added to each well. Samples were immediately subjected to FACS analysis using a BD FACS Canto™ II Flow Cytometer. The degree of apoptosis was defined of the percentage of Annexin V positive cells of total cells.

Statistical Analysis: Evaluation of Combination Effect

A statistical analysis was carried out with the software product SAS (SAS Institute, Cary, N.C. USA), version 9.2. The statistical analysis was performed separately for each chemotherapeutic agent within each cell line. Summary statistics (number of observations, mean, median, standard deviation and coefficient of variation) were calculated per experimental group for each assay individually as well as for the pooled data of all three assays. The data were analyzed by a one-factorial analysis of variance (ANOVA) followed by estimating and two-sided testing of interesting linear contrast for deviation from zero. Contrasts were defined to investigate pairwise comparisons as well as the question whether the combination therapy results in more (or less) than additive effects (interaction contrasts). Effects were quantified by mean differences and their two-sided 95% confidence intervals. Interaction contrasts were considered synergistically for p-values<0.05.

### EXAMPLES

### Example 1

### Pro-Apoptotic Effect of mAb A2 in Combination with Bendamustine

Ramos and Raji Burkitt lymphoma cells were incubated for 48 hrs with mAb A2 at a concentration of 10 μg/ml, bendamustine at concentrations of 100 μM, 200 μM and 400 μM, or combinations thereof. Three independent experiments were performed for each cell line. The mean apoptosis induction is shown in FIG. 1 and FIG. 2. Mab A2 alone induced apoptosis in 12% of Raji cells and 9% of Ramos cells, respectively. Single agent bendamustine caused 10% (200 μM) and 13% (400 μM) apoptosis on Raji cells and 19% (100 μM) and 35% (400 μM) apoptosis on Ramos cells. The combination of mAb A2 with bendamustine induced significantly greater apoptosis than treatment with single agents. On Raji cells, the combination of mAb A2 with 200 μM bendamustine resulted in 35% apoptotic cells, the combination of mAb A2 with 400 μM bendamustine resulted in 37% apoptotic cells. On Ramos cells, the combination of mAb A2 with 100 μM bendamustine resulted in 50% apoptotic cells, the combination of mAb A2 with 400 μM bendamustine resulted in 73% apoptotic cells. The pro-apoptotic effect of the combination was surprisingly higher than the calculated additive effect of both individual treatments (FIGS. 1 and 2). Statistical analysis of interaction contrasts showed significant differences between the single agents and the combination groups, indicating synergistic activity of mAb A2 in combination with bendamustine (FIG. 3).

Apoptosis Induction on Raji Cells

Bendamustine concentration

| | |
|---|---|
| 200 μM | 400 μM |

% apoptotic cells

| | | |
|---|---|---|
| A2 10 μg/ml | 12 | 12 |
| Bendamustine | 10 | 13 |
| A2 + bendamustine calculated | 21 | 25 |
| A2 + bendamustine measured | 35 | 37 |

Apoptosis Induction on Ramos Cells

Bendamustine concentration

| | |
|---|---|
| 100 μM | 400 μM |

% apoptotic cells

| | | |
|---|---|---|
| A2 10 μg/ml | 9 | 9 |
| Bendamustine | 19 | 35 |
| A2 + bendamustine calculated | 29 | 45 |
| A2 + bendamustine measured | 50 | 73 |

### Example 2

### Anti-Tumor Effect of mAb A2 in Combination with Bendamustine in a Human Xenograft Tumor Model

Human xenograft tumor models are utilized to assess the efficacy of anti-cancer agents against human tumor cells in immunocompromised mice. DoHH2 tumor cells are a CD37 positive B-lymphoblastoid cell line derived from a patient with a follicular B-cell lymphoma. The tumor cells are engrafted s.c. into the left or right flank of CB-17 SCID mice, e.g. by injecting 1×10⁷ tumor cells in a volume of 100 μl via a syringe. Tumor growth is monitored three times a week by measurement of tumor volumes using a caliper. After tumors have reached a certain size, e.g. 100 mm³, animals are randomized into different groups of 10 animals per group and are treated with antibody A2, bendamustine, or a combination thereof. Vehicle treated mice serve as a control for tumor growth. Mice are treated with antibody A2 at a dose of 10 mg/kg twice weekly, bendamustine 10 mg/kg twice weekly ip, or a combination thereof.

Control treated animals display a rapid tumor growth and are sacrificed after 2 to 3 weeks after start of treatment when tumors have reached a critical tumor size of 1500 mm³. mAb A2 and bendamustine single agent treatment show a significant anti-tumor effect, e.g. tumor growth retardation, compared to control treated animals. The combination of mAb A2 and bendamustine shows a significantly improved anti-tumor effect over that of single agent treatment. These results indicate that a combination of mAb A2 with bendamustine results in superior anti-tumor efficacy compared to single agent treatment.

### Example 3

### Efficacy of mAb A2 in Combination with Bendamustine Chemotherapy in a Mouse Model of Human Follicular Lymphoma

Objectives of the Study

The goal of the present study was to assess the efficacy of antibody A2 in combination with bendamustine chemotherapy in a model of human follicular lymphoma (DOHH2) in C.B-17 scid mice.

Design of the Study

| | Number | | Dose | Schedule [days of | |
|---|---|---|---|---|---|
| Group | of mice | Compound | [mg/kg] | admin. per week] | Route |
| 1 | 7 | NaCl (0.9%) | — | d 1, d 5, d 8, d 12, | i.p. |
| | | | | d 15 | |
| 2 | 7 | antibody A2 | 10 | d 1, d 5, d 8, d 12, | i.p. |
| | | | | d 15 | |
| 3 | 7 | bendamustine | 20 | d 2, d 4 | i.p. |
| 4 | 7 | antibody A2 + | 10 + 20 | d 1, d 5, d 8, d 12, | i.p. |
| | | bendamustine | | d 15 d 2, d 4 | |

Materials and Methods

A single batch of antibody A2 was used for this study. Bendamustine (Ribomustine®) was purchased from MundiPharma. Female C.B-Igh-1^{b}/IcrTac-Prkdc^{scid} mice were used. Antibody A2 and bendamustine were administered twice weekly intraperitoneally. Tumors were established from cultured DOHH2 cells by subcutaneous injection. Tumor volumes were determined three times a week using a caliper. Body weight of the mice was measured as an indicator of tolerability of the compounds on the same days. Day 1 was the first, day 16 the last day of the study.

Main Results

The following tables summarize the results obtained for tumor volume and body weight after two cycles of therapy (day 16 of the study).

| | TGI | | Weight change | |
|---|---|---|---|---|
| Compound | [%] | p value | [%] | p value |
| Vehicle control | — | — | +4.6 | — |
| 10 mg/kg antibody A2 | 73 | 0.0009 | +3.1 | 0.8048 |
| 20 mg/kg bendamustine | 57 | 0.0014 | +2.4 | 0.0530 |
| 10 mg/kg antibody A2 + | 105 | 0.0014 | −7.6 | 0.0006 |
| 20 mg/kg bendamustine | | | | |

| | 10 mg/kg | p value vs | Combi- | p value vs | 20 mg/kg |
|---|---|---|---|---|---|
| | antibody | combination | nation | combination | benda- |
| | A2 | therapy | therapy | therapy | mustine |
| TGI [%] | 73 | 0.0014 | 105 | 0.0014 | 57 |
| PR [x/7] | 0 | — | 1 | — | 0 |
| CR [x/7] | 0 | — | 6 | — | 0 |
| Weight | +3.1 | 0.0012 | −7.6 | 0.0023 | −5.4 |
| change | | | | | |
| [%] | | | | | |

Conclusions

Antibody A2 as a single agent significantly inhibited growth of DOHH2 follicular lymphoma and was well tolerated. Bendamustine administered as a single agent showed significant inhibition of tumor growth but resulted in weight loss. The combination of antibody A2 and bendamustine was significantly more efficacious than either monotherapy, inducing tumor regression in all animals. Statistical analysis showed synergism of the combination treatment. Body weight loss was slightly higher than with bendamustine alone.

1. Introduction

Antibody A2 is a mouse-human chimeric Fc-engineered IgG1 antibody with high affinity for CD37 and potent in vitro cytotoxicity (apoptosis, ADCC, tumor cell depletion in whole blood assays). The goal of the present study was to assess the efficacy of antibody A2 in combination with bendamustine chemotherapy in a model of human follicular lymphoma (DOHH2) in C.B-17 scid mice

1.1 Study Design
Model: Subcutaneous xenografts of the human Burkitt lymphoma (Ramos) growing in nude mice

| | Number | | Dose | Schedule [days of | |
|---|---|---|---|---|---|
| Group | of mice | Compound | [mg/kg] | administration] | Route |
| 1 | 7 | NaCl (0.9%) | — | d 1, d 5, d 8, d 12, | i.p. |
| | | | | d 15 | |
| 2 | 7 | Antibody A2 | 10 | d 1, d 5, d 8, d 12, | i.p. |
| | | | | d 15 | |
| 3 | 7 | bendamustine | 20 | d 2, d 6 | i.p. |
| 4 | 7 | Antibody A2 + | 10 + 20 | d 1, d 5, d 8, d1 2, | i.p. |
| | | bendamustine | | d 15 d 2, d 6 | |

1.2 Test Compounds

Antibody A2 (10 mg/ml) was used for this experiment and formulated in a vehicle containing 25 mM Na-citrate, 115 mM NaCl and 0.04% Tween 80, pH 6.0 and diluted with PBS. Bendamustine (Ribomustine®) was purchased from MundiPharma and dissolved in Ampuwa (water for injection) and adjusted to pH5 using NaOH.

1.3 Mice

Mice were 6 week-old female C.B-Igh-1^{b}/IcrTac-Prkdc^{scid} purchased from Taconic, Denmark. After arrival, mice were allowed to adjust to ambient conditions for at least 5 days before they were used for the experiments. They were housed in Makrolon® type III cages in groups of 7 under standardized conditions at 21.5±1.5° C. temperatures and 55±10% humidity. Standardized diet (PROVIMI KLIBA) and autoclaved tap water were provided ad libitum. Subcutaneously implanted (under isoflurane anesthesia) microchips were used to identify each mouse. Cage cards showing the study number, the animal identification number, the compound and dose level, the administration route as well as the schedule remained with the animals throughout the study.

1.4 Establishment of Tumors, Randomization

To establish subcutaneous tumors, DOHH2 cells were harvested by centrifugation, washed and resuspended in PBS+5% FCS at 1×10⁸ cells/ml. 100 μl cell suspension containing 1×10⁷ cells was then injected subcutaneously into the right flank of the mice (1 site per mouse). Mice were randomly distributed between the treatment and the vehicle control group (10 days after cell injection) when tumors were well established and had reached volumes of 34 to 100 mm³.

1.5 Administration of Test Compound

Antibody A2 was diluted with PBS and injected intraperitoneally with a volume of 10 ml/kg.

Bendamustine was diluted with Ampuwa (water for injection) and injected intraperitoneally with a volume of 10 ml/kg. Solutions were kept at 6° C. for a maximum of 5 days.

1.6 Monitoring Tumor Growth and Side Effects

Tumor diameters were measured three times a week (Monday, Wednesday and Friday) with a caliper. The volume of each tumor [in mm³] was calculated according to the formula “tumor volume=length*diameter²*π/6.” To monitor side effects of treatment, mice were inspected daily for abnormalities and body weight was determined three times a week (Monday, Wednesday and Friday). Animals were sacrificed when the control tumors reached a size of approximately 1000 mm³ on average. In addition, animals with tumor sizes exceeding 1.5 cm in diameter or 20% body weight loss were euthanized for ethical reasons.

TGI values were calculated as follows:

TGI=100×{1−[(treated_{final day}−treated_{day1})/(control_{final day}−control_{day1})]}

1.7 Statistical Analysis

1.7.1 Anti-Tumor Efficacy and Change of Body Weight

For the evaluation of the statistical significance of tumor inhibition a one-tailed non-parametric Mann-Whitney-Wilcoxon U-test was performed, based on the hypothesis that an effect would only be measurable in one direction (i.e. expectation of tumor inhibition but not tumor stimulation). In general, the U-test compares the ranking of the individual tumors of two groups, according to (in this study) absolute volume on a particular day (pairwise comparisons between groups). Analysis was performed on the last days of the experiment. Tumors to which the LOCF methodology was applied until the day of the statistical analysis were included in the comparison. The p-values obtained from the U-test were adjusted using the Bonferroni-Holm correction. By convention, p-values≦0.05 indicate significance of differences. Statistical calculations were performed using GraphPad Prism Bioanalytic Software (version 5.04 for Windows, GraphPad Software, San Diego Calif. USA, www.graphpad.com).

1.7.2 Synergism of Efficacy

The statistical evaluation was performed for the parameter tumor volume at different days. All measurements were available for all treatment groups up to Days 16, i.e. there are no missing values up to Day 16.

The tumor volume was analyzed based on descriptive statistics and by using a Mixed Model for Repeated Measurements (MMRM) up to 16 days.

The number of valid observations, mean, standard deviation, median, and the geometric mean were given. Data were log-transformed to stabilize the variance over the time course. Concerning the data on the log-scale (natural logarithm, i.e. logarithm to the base e) mean, standard deviation, median, minimum and maximum were displayed.

All statistical analyses were exploratory, no adjustment of the significance level for multiple testing was made, i.e., all p-values reported will have to be interpreted as part of the descriptive and exploratory analyses.

After data screening, it was noticed that linearity described sufficiently well the logarithmized tumor volume dynamic within an animal up to day 16. The repeated tumor volume measurements were analyzed after log-transformation by a linear mixed effects model for repeated measurements. For tumor volumes measured as 0 mm³ the undefined logarithm was set to 0, corresponding to a tumor volume of 1 mm³ on the original scale. Treatment, time, and interaction term treatment*time were included as fixed effects and animal was considered as a random effect. The log-transformed tumor volume at baseline was included as a covariate in the model

Yᵢⱼₖ=μ+αᵢ+dᵢⱼ−τₖ+(ατ)ᵢₖ+βχᵢⱼ+εᵢⱼₖ.

Where Yᵢⱼₖ is the log-transformed tumor volume at time k on animal j in treatment group i, μ is the overall mean, αᵢ is a fixed effect of treatment i, dᵢⱼ is a random effect of animal j in treatment group i, τₖ is fixed effect of time k, (ατ)ᵢₖ is a fixed interaction effect of treatment i with time k, χᵢⱼ is the log-transformed tumor volume at baseline as a covariable, and εᵢⱼₖ is random error at time k on animal j in treatment i.

Regarding the within-subject covariance matrix R, a variance components (VC) covariance matrix R(i,j)=σₖ²χ(i=j) was chosen. The VC structure was also indicated as best among of some reasonable covariance structures on the basis of the AIC criterion. An unstructured covariance (UN) matrix has not led to a positive definite Hessian matrix and could not be considered.

The covariance parameters were estimated using residual (restricted) maximum likelihood (REML). The Kenward Roger (KR) method was chosen as the denominator degrees of freedom option in SAS PROC MIXED procedure. KR works reasonably well also with more complicated covariance structures, when sample sizes are moderate to small and the design is reasonably balanced.

To assess antagonism or synergism, additive treatment effects were calculated as summation of the monotherapy effects on log-scale (log μ_{Ref}−log μ_{T}_{₁}+log μ_{Ref}−log μ_{T}_{₂}) and were compared with the effect of the corresponding combination therapy (log μ_{Ref}−log μ_{T}_{₁}_{T}_{₂}).

The statistical evaluation was prepared using the software package SAS version 9.2 (SAS Institute Inc., Cary N.C., USA).

2. Results

2.1 Tumor Volume and Body Weight: Treatment vs Control

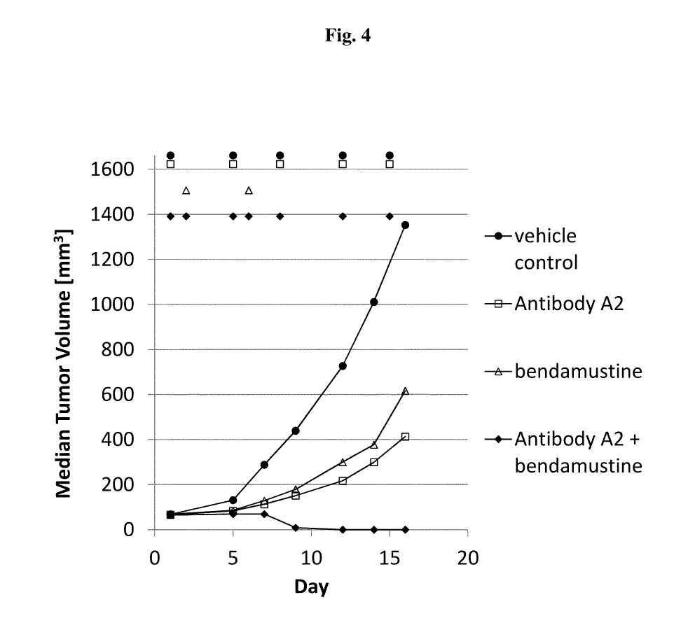

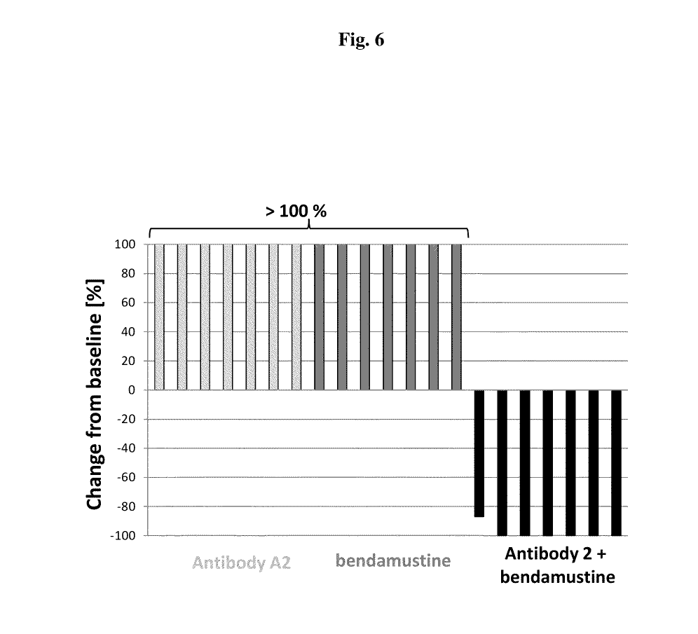

During the 16 day treatment period, control tumors grew from a median volume of 70 mm3 to a volume of 1330 mm3 (FIG. 4, Table 1). The control animals gained 3.7% body weight (FIG. 6, Table 1).

Treatment with 1 mg/kg antibody A2 twice weekly intraperitoneally for two weeks significantly delayed tumor growth compared to the controls (median TGI=73%, p=0.0009) (FIG. 4, Table 1). Similar gain of body weight was observed compared to vehicle-treated control animals (+3.1%; p=0.8048, not significant) (FIG. 6, Table 1).

Treatment with bendamustine administered twice (day 2 and 6) i.p. significantly delayed tumor growth compared to the controls (median TGI=57%, p=0.0014) (FIG. 4, Table 1). The body weight gain was not significantly different compared to the vehicle-treated control animals (+2.4%; p=0.0530) (FIG. 6, Table 1). Because body weight loss of up to 7.9% in median appeared within the first week of treatment, the treatment was stopped after the first cycle of two injections.

Treatment with the combination of antibody A2 and bendamustine significantly delayed tumor growth compared to the controls (median TGI=105%, p=0.0014) (FIG. 6, Table 1). The loss of body weight was significantly different compared to the weight gain of the vehicle-treated control animals (−7.6%; p=0.0006) (FIG. 6, Table 1). Because body weight loss appeared within the first week of treatment, the treatment with bendamustine was stopped after the first cycle of two injections.

2.2 Tumor Volume and Body Weight: Combination Therapy vs Single-Agent Therapy

Two cycles of therapy with a combination of antibody A2 and bendamustine was significantly more efficacious (median TGI=105%, p=0.0014) than the single agent treatment with antibody A2 (median TGI=73%, p=0.0014) or with bendamustine (median TGI=57%, p=0.0014) (FIG. 4, Table 2).

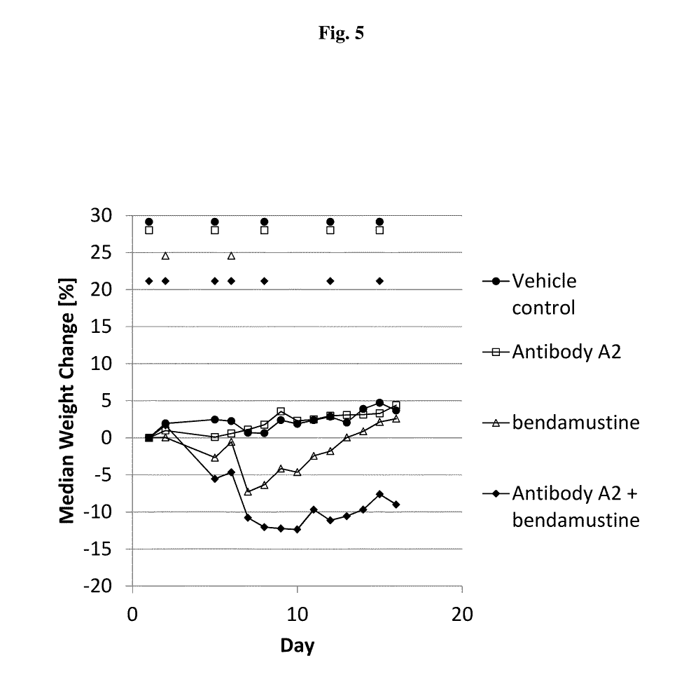

All 7 tumors each treated with either antibody A2 or bendamustine were growth inhibited while 6 out of 7 tumors treated with the combination completely regressed and one out of 7 partially regressed to a volume of only 9 mm3 (FIG. 5, Table 2).

Superiority of the combination therapy with antibody A2 and bendamustine compared to the additive effect of the corresponding monotherapies could be detected statistically from day 9 on (p<0.0001).

On day 16, body weight loss in the combination group (−7.6%) was significantly different compared to single-agent antibody A2 (+3.1%, p=0.0012) and bendamustine (+2.4%, p=0.0023) (FIG. 6, Table 2).

3. Discussion

In the present study, two cycles of treatment with antibody A2 at a dose of 10 mg/kg twice weekly significantly delayed tumor growth (TGI=72%). Treatment was well tolerated, One cycle of bendamustine treatment resulted in significant efficacy (TGI=57%) and was moderately tolerated.

Two cycles of combination therapy with antibody A2 and one cycle of bendamustine showed improved efficacy (TGI=105%, 6 out of 7 complete and one partial tumor regressions) compared to single-agent treatment resulting in synergism. Combination treatment was considered to be moderately tolerated by the animals.

4. Conclusion

Antibody A2 as a single agent significantly inhibited growth of DOHH2 follicular lymphoma and was well tolerated. Bendamustine administered as a single agent showed significant inhibition of tumor growth but resulted in weight loss. The combination of Antibody A2 and bendamustine was significantly more efficacious than either monotherapy, inducing tumor regression in all animals. Statistical analysis showed synergism of the combination treatment. Body weight loss was slightly higher than with bendamustine alone.

TABLE 1
Tumor volume and body weight: treatment
vs. control (results on day 16)

| | TGI | | Weight change | |
|---|---|---|---|---|
| Compound | [%] | p value | [%] | p value |
| Vehicle control | — | — | +4.6 | — |
| 10 mg/kg Antibody A2 | 73 | 0.0009 | +3.1 | 0.8048 |
| 20 mg/kg bendamustine | 57 | 0.0014 | +2.4 | 0.0530 |
| 10 mg/kg Antibody A2 + | 105 | 0.0014 | −7.6 | 0.0006 |
| 20 mg/kg bendamustine | | | | |

bold p value < 0.05

TABLE 2
Tumor volume and body weight: combination therapy
vs. single agent therapy (results on day 16)

| | 10 mg/kg | p value vs | Combi- | p value vs | 20 mg/kg |
|---|---|---|---|---|---|
| | Antibody | combination | nation | combination | benda- |
| | A2 | therapy | therapy | therapy | mustine |
| TGI [%] | 73 | 0.0014 | 105 | 0.0014 | 57 |
| PR [x/7] | 0 | — | 1 | — | 0 |
| CR [x/7] | 0 | — | 6 | — | 0 |
| Weight | +3.1 | 0.0012 | −7.6 | 0.0023 | −5.4 |
| change | | | | | |
| [%] | | | | | |

bold p value < 0.05

What is claimed is:

1. A method of using a CD37 antibody in combination with bendamustine for the treatment of a patient suffering from a CD37-positive malignancy, whereby the CD37 antibody comprises:
a variable heavy chain comprising CDRs having the SEQ ID NOs: 15, 16 or 21, and 17, and
a variable light chain comprising CDRs having the SEQ ID NOs: 18, 19 and 20.

2. The method of claim 1, wherein the patient receives at least one dose of the CD37 antibody and at least one dose of bendamustine during a treatment cycle, whereby a treatment cycle is a time period of about 1 to 6 weeks.

3. The method of claim 1, whereby the CD37 antibody is administered to said patient simultaneously with the administration of bendamustine.

4. The method of claim 1, whereby the CD37 antibody is administered to said patient at least 24 hrs after the administration of bendamustine.

5. The method of claim 1, whereby the CD37 antibody is administered to said patient within 36 hrs after the administration of bendamustine.

6. The method of claim 1, whereby the CD37 antibody is administered to said patient after a 2 day consecutive application of bendamustine, and within 36 hrs after the administration of the second bendamustine dosage.

7. The method of claim 1, whereby the CD37 antibody is administered to said patient before a 2 day consecutive application of bendamustine, and within 36 hrs before the administration of the first bendamustine dosage.

8. The method of claim 1, whereby the CD37 antibody is additionally administered at least one more time during a treatment cycle, and in the middle of the treatment cycle at about 2 weeks or once weekly.

9. The method of claim 1, whereby the said CD37 antibody is administered in a dose of about 0.01 μg/kg to 40 mg/kg.

10. The method of claim 1, whereby the dose for a 70 kg human is from 1 mg to 2800 mg weekly or 2 mg to 800 mg every 2 weeks, whereby the CD37 antibody comprises SEQ ID NOs: 5 and 6.

11. The method of claim 1, whereby the dose for a 70 kg human is from 1 mg to 2800 mg weekly or 200 mg to 770 mg every two weeks, whereby the CD37 antibody comprises SEQ ID NOs:11 and 12.

12. The method of claim 1, whereby the dose for bendamustine is from 50 to 150 mg/m² body surface.

13. The method of claim 1, whereby the patient is a patient suffering from chronic lymphocytic leukemia (CLL) and whereby bendamustine is administered at a dosage of 100 mg/m² body surface on days 1 and 2 of the treatment cycle and for a period of 3 to 4 weeks.

14. The method of claim 1, whereby the patient is a patient suffering from B-cell non-Hodgkin's lymphoma (B-NHL) and whereby bendamustine is administered at a dosage of 120 mg/m² body surface on days 1 and 2 of the treatment cycle, and for a period of 3 to 4 weeks.

15. The method of claim 1, whereby bendamustine is administered as a one-time administration per treatment cycle at a dose of 70 to 400 mg/m² body surface.

16. The method of claim 1, whereby the combination of the CD37 antibody and bendamustine is administered as first line treatment.

17. The method of claim 1, whereby the combination of the CD37 antibody and bendamustine is administered as second or later line treatment.

18. The method of claim 1, whereby the CD37-positive malignancy is selected from the group consisting of: multiple myeloma, plasmacytoma, T-cell lymphoma, acute lymphoblastic leukemia (ALL), and B-cell malignancies, e.g. B-cell lymphomas, agressive B-cell lymphoma, Hodgkin's disease, B-cell non-Hodgkin's lymphoma (NHL), lymphomas, Waldenström's macroglobulinaemia, central nervous system lymphomas, leukemias, acute lymphoblastic leukemia (ALL), chronic lymphocytic leukemia (CLL), hairy cell leukemia, chronic myoblastic leukemia, small lymphocytic lymphoma, B-cell prolymphocytic leukemia, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt's lymphoma/leukemia, grey zone lymphoma, B-cell proliferations of uncertain malignant potential, lymphomatoid granulomatosis, and post-transplant lymphoproliferative disorder.

19. A method of reducing CD37-positive cells comprising:
a) exposing CD37-positive cells to a CD37 antibody and
b) exposing CD37-positive cells to bendamustine,
whereby said CD37 antibody of step a) comprises:
i) a variable heavy chain comprising CDRs have the SEQ ID NOs: 15, 16 or 21, and 17, and
ii) a variable light chain comprising CDRs having the SEQ ID NOs: 18, 19 and 20.

20. The method of claim 19, whereby the CD37-positive cells are exposed to the CD37 antibody and bendamustine simultaneously.

21. The method of claim 19, whereby the CD37-positive cells are exposed to the CD37 antibody at least 24 hrs after they are exposed to bendamustine.

22. The method of claim 19, whereby the CD37-positive cells are exposed to the CD37 antibody within 36 hrs after they are exposed to bendamustine.

23. A method of treating a CD37-positive malignancy, comprising administrating a therapeutically effective amount of i) a CD37 antibody and ii) bendamustine to a patient in need thereof, whereby the CD37 antibody comprises:
a) a variable heavy chain comprising CDRs have the SEQ ID NOs: 15, 16 or 21, and 17, and
b) a variable light chain comprising CDRs having the SEQ ID NOs: 18, 19 and 20.

24. A pharmaceutical composition comprising a CD37 antibody, bendamustine, and a pharmaceutically acceptable carrier, whereby the CD37 antibody comprises:
a) a variable heavy chain comprising CDRs have the SEQ ID NOs: 15, 16 or 21, and 17, and
b) a variable light chain comprising CDRs having the SEQ ID NOs: 18, 19 and 20.
